(19) United States
(12) Patent Application Publication
(10) Pub. No.: US 2022/0207424 A1
(43) Pub. Date: Jun. 30, 2022

(54) ADAPTIVE TRAINING METHOD OF A BRAIN COMPUTER INTERFACE USING A PHYSICAL MENTAL STATE DETECTION

(71) Applicant: COMMISSARIAT A L'ENERGIE ATOMIQUE ET AUX ENERGIES ALTERNATIVES, Paris (FR)

(72) Inventors: Vincent ROUANNE, Grenoble Cedex 09 (FR); Tetiana AKSENOVA, Grenoble Cedex 09 (FR)

(73) Assignee: COMMISSARIAT A L'ENERGIE ATOMIQUE ET AUX ENERGIES ALTERNATIVES, Paris (FR)

(21) Appl. No.: 17/563,700

(22) Filed: Dec. 28, 2021

(30) Foreign Application Priority Data
Dec. 29, 2020 (FR) 20 14199

(51) Int. Cl. G06N 20/00; G06N 5/04

(52) U.S. Cl. CPC G06N 20/00; G06N 5/04

(57) ABSTRACT

The present invention relates to an adaptive training method of a brain computer interface. The ECoG signals expressing the neural command of the subject are preprocessed to provide at each observation instant an observation data tensor to a predictive model that deduces therefrom a command data tensor making it possible to control a set of effectors. A satisfaction/error mental state decoder predicts at each epoch a satisfaction or error state from the observation data tensor. The mental state predicted at a given instant is used by an automatic data labelling module to generate on the fly new training data from the pair formed by the observation data tensor and the command data tensor at the preceding instant. The parameters of the predictive model are subsequently updated by minimising a cost function on the training data thus generated.

### TECHNICAL FIELD

The present invention relates to the field of Brain Computer Interfaces (BCI) or Brain Machine Interfaces (BMI). It particularly applies to the direct neural command of a machine, such as an exoskeleton or a computer.

### PRIOR ART

Brain computer interfaces use the electrophysiological signals emitted by the cerebral cortex to develop a command signal. These neural interfaces have been the subject of much research particularly in the aim of restoring a motor function in a paraplegic or tetraplegic subject with the aid of a prosthesis or of a motorised orthosis.

Neural interfaces may be of invasive or non-invasive nature. Invasive neural interfaces use intracortical electrodes (that is to say implanted in the cortex) or cortical electrodes (disposed at the surface of the cortex) collecting in the latter case electrocorticography (ECoG) signals. Non-invasive neural interfaces use electrodes placed on the scalp to collect electroencephalography (EEG) signals. Other types of sensors have also been envisaged such as magnetic sensors measuring the magnetic fields induced by the electrical activity of the neurons of the brain. Therefore, we speak of magnetoencephalography (MEG) signals.

Advantageously, brain computer interfaces use ECoG type signals, having the advantage of a good compromise between biocompatibility (array of electrodes implanted at the surface of the cortex) and quality of the signals collected.

The ECoG signals thus measured must be processed in order to estimate the trajectory of the movement desired by the subject and deduce therefrom the command signals of the computer or of the machine. For example, when this involves commanding an exoskeleton, the BCI estimates the trajectory of the desired movement from the electrophysiological signals measured and deduces therefrom the control signals making it possible for the exoskeleton to reproduce the trajectory in question. Similarly, when this involves commanding a computer, the BCI estimates for example the desired trajectory of a pointer or of a cursor from the electrophysiological signals and deduces therefrom the command signals of the cursor/pointer.

The trajectory estimation, and more specifically that of the kinematic parameters (position, speed, acceleration), is also named neural decoding in the literature. Neural decoding particularly makes it possible to command a movement (of a prosthesis or of a cursor) from ECoG signals.

The trajectory estimation and the computation of the control signals of the exoskeleton or of the effector generally requires a training or calibration phase beforehand, known as off-line. During this phase, the subject imagines, observes or performs a movement according to a determined trajectory during a given calibration interval. The electrophysiological signals measured during this interval are exploited in relation to this trajectory to construct a predictive model and more specifically to compute the parameters of this model.

The validity of the predictive model is however limited over time due to the non-stationary condition of the neural signals. For this reason, it is necessary to carry out an on-line calibration of the predictive model, that is to say as the neural signals are observed and the command applied.

An on-line calibration method of a BCI has been described in the article by A. Eliseyev et al. entitled “Recursive exponentially weighted N-way Partial Least Squares regression with recursive validation of hyper-parameters in Brain-Computer Interface applications” published in Scientific Reports, vol. 7, no. 1, p. 16281, November 2017 as well as in the patent application FR-A-3 061 318. This method will be designated in the following under the acronym REW-NPLS (Recursive Exponentially Weighted N-way Partial Least Squares).

Due to the non-stationary condition of neural signals, dedicated on-line calibration sessions must be periodically planned to train the predictive model of the BCI. The usage and calibration phases of the BCI are mutually exclusive from one another as explained in relation to FIGS. 1A and 1B.

FIG. 1A schematically represents the operation of a brain computer interface trained beforehand.

The ECoG signals of the subject are captured and submitted for processing in a preprocessing module 110 to provide an observation data tensor of order P, denoted [symbol] , where t represents an observation instant. The observation data tensor is generally of dimension

[symbol]

The observation tensor is subsequently provided as input tensor for a predictive module 120 trained beforehand. The latter predicts, from the input tensor, an output tensor (or command tensor) of order [symbol] , denoted [symbol] . The output tensor is generally of dimension J₁× . . . J_{M}. The instant t as index is that at which the command is applied, the command data being able to correspond to various effectors, schematically represented as 130, or to various degrees of freedom of a multi-axis robot for example.

These various effectors make it possible to move an exoskeleton (or a multi-axis robot), represented as 140. The movement of the exoskeleton generates a sensory (visual for example) counter-reaction with the subject which translates by the generation of new ECoG signals.

A training session in supervised mode of a brain computer interface has been schematically represented in FIG. 1B.

It is recognised in FIG. 1B the preprocessing module 110, the predictive model 120, the effectors 130 and the exoskeleton 140.

In this training (or calibration) phase of the BCI, the subject is requested to carry out a predetermined task (for example carry out a movement). The observation data (represented in a synthetic manner by the observation tensor) from the preprocessing module are labelled with labels associated with the command data (represented in a synthetic manner by a command tensor) corresponding to the execution of the task by the effectors. Thus, the predictive module is trained to deduce from observation data associated with the task in question (predictive variables) the command data making it possible to perform this task (target variables). The predictive model may particularly be trained by means of the REW-NPLS algorithm mentioned above.

Consequently, it is understood that the training phases do not make it possible to freely use the BCI. The interruption of this free use by dedicated training phases is highly detrimental in terms of availability and practicality.

The object of the present invention is consequently to propose a brain computer interface that can adapt to the non-stationary condition of the neural signals while preventing the interruption of its use by dedicated training phases.

### DESCRIPTION OF THE INVENTION

The present invention is defined by a method for training a brain computer interface intended to receive a plurality of electrophysiological signals expressing a neural command of a subject, during a plurality of observation windows associated with observation instants, said electrophysiological signals being preprocessed in a preprocessing module to form at each observation instant an observation data tensor, the brain computer interface using a predictive model to deduce at each observation instant a command data tensor from the observation data tensor, said command data being intended to control at least one effector to perform a trajectory, said training method being original in that:
at each observation instant, a satisfaction/error mental state of the subject is decoded from the observation data tensor by means of a decoder trained beforehand, said mental state being representative of the conformity of the trajectory with the neural command;
training data are generated from the satisfaction/error mental state decoded at a given observation instant, and from the pair formed by the observation data tensor and the command data tensor at a preceding instant;
the parameters of the predictive model are updated by minimising a cost function on the training data generated at the preceding step.

The mental state decoder is advantageously trained in a previous phase by presenting simultaneously to the subject a movement setpoint and a trajectory, the observation data tensor being labelled with a satisfaction mental state when the trajectory is in accordance with the setpoint and with an error mental state when it deviates therefrom.

The mental state decoder typically provides at each observation instant a prediction of the mental state in the form of a binary value (ŷ_{D,mental_state}^{t}) as well as an estimation of the degree of certainty of this predition (|ŷ_{mental_state}^{t}|).

According to a first embodiment, the prediction made by the predictive model is based on a classification, the command data tensor being obtained from the most probable class predicted by the predictive model.

In this case, if the mental state predicted at an observation instant is a satisfaction state, the training data may only be generated from the observation data tensor and from the command data tensor at the preceding observation instant, if the degree of certainty of the predicted mental state is greater than a first predetermined threshold value (Th_{mental_state}¹).

If the mental state predicted at an observation instant is an error state, the training data may only be generated from the observation data tensor and from the command data tensor at the preceding observation instant, if the degree of certainty of the predicted mental state is greater than a second predetermined threshold value (Th_{mental_state}²).

According to the first embodiment, if the mental state predicted at an observation instant is an error state, the training data generated comprise the observation data tensor at the preceding observation instant as well as a command data tensor obtained from the second most probable class predicted by the predictive model at the preceding observation instant.

The cost function used for updating the parameters of the predictive model advantageously expresses the square deviation between the command data tensor predicted by the model and that provided by the training data, said square deviation being weighted by the degree of certainty predicted by the mental state decoder during the generation of these training data, the square deviation thus weighted being added up on the training data set.

According to a second embodiment, the prediction made by the predictive model is based on a linear or multilinear regression.

According to a first variant, if the mental state predicted at an observation instant is an error state, the training data are not generated if this predicted mental state is a satisfaction state, the training data are only generated from the observation data tensor and from the command data tensor at the preceding observation instant, if the degree of certainty of the predicted mental state is greater than the first predetermined threshold value (Th_{mental_state}¹).

According to a second variant, regardless of the state predicted at an observation instant, the training data are generated from the observation data tensor and from the command data tensor at the preceding observation instant, the training data then being associated with the degree of certainty of the prediction of the predicted mental state (|ŷ_{mental_state}^{t}|).

The cost function used for updating the parameters of the predictive model advantageously depends on the square deviation between the command data tensor predicted by the predictive model and that provided by the training data, this dependency with the square deviation being increasing when the mental state predicted during the generation of the training data was a satisfaction state and decreasingly when this mental state was an error signal, said square deviation being weighted by a factor depending increasingly on the degree of certainty of the predicted mental state, associated with the training data.

### BRIEF DESCRIPTION OF THE FIGURES

Other features and advantages of the invention will become apparent upon reading a preferable embodiment of the invention, described with reference to the appended figures, wherein:

FIG. 1A schematically represents the operation of a brain computer interface trained beforehand;

FIG. 1B schematically represents a supervised training session of a brain computer interface;

FIG. 2 schematically represents the operation of an adaptive brain computer interface according to one embodiment of the present invention using a first type of architecture;

FIG. 3 schematically represents the operation of an adaptive brain computer interface according to one embodiment of the present invention using a second type of architecture.

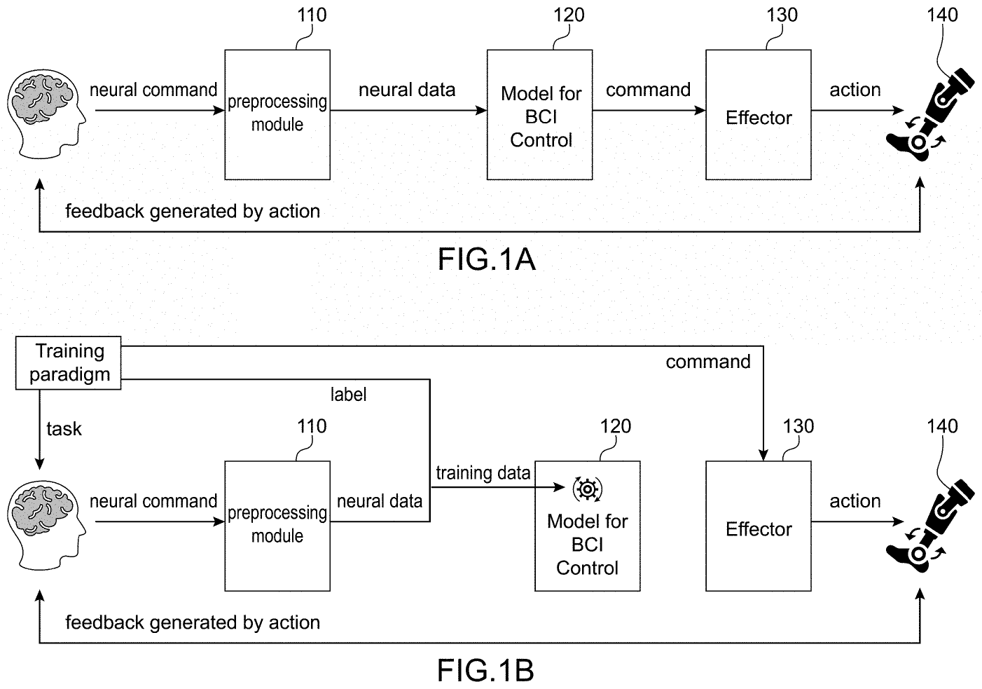

### DESCRIPTION OF EMBODIMENTS

It will be considered in the following a brain computer interface (BCI) such as presented in the introductory part.

The electrophysiological signals from the various electrodes are sampled and assembled by data blocks, each block corresponding to an observation sliding window of width ΔT. Each observation window is defined by an observation instant or epoch at which the window in question starts.

The electrophysiological signals may be subject to a preprocessing. This preprocessing may particularly include an elimination of the average taken on the set of electrodes, then a time-frequency analysis is carried out on each of the observation windows.

The time-frequency analysis may be based on a breakdown into wavelets, for example into Morlet wavelets or a CCWT (Continuous Complex Wavelet Transform) breakdown. The person skilled in the art will nevertheless understand that other types of time-frequency analysis may be envisaged by the person skilled in the art.

These results of the time-frequency analysis may further be subject to a frequency smoothing or a decimation.

Thus, an observation data tensor of order 3, denoted [symbol] , is associated with each observation window, or observation instant t, of which the first mode corresponds to the temporal positions of the wavelets, the second mode corresponds to the frequency, in other words to the number of frequency bands used for the breakdown into wavelets on an observation window and the fourth mode corresponds to the space, in other words to the sensors (electrodes). Thus, [symbol] ∈ [symbol] ^{τ×f×s} and the complete tensor of the observation data, that is to say the history of observations, is denoted X∈ [symbol] where N is the number of epochs, τ is the number of temporal positions of the wavelets (temporal features), if applicable after averaging on a plurality of successive temporal positions, f is the number of frequency bands (frequency features), and s is the number of sensors (spatial features). More generally, the observation data tensor, [symbol] , relating to the epoch t may be of order P. In this case, the observation tensor [symbol] is of dimension I₁× . . . ×I_{P}. Nevertheless, without loss of generality, the invention will be described in the aforementioned case P=3.

In the same way, the trajectory of the movement imagined, observed or performed at the instant may be described by an output tensor (or command tensor) of order [symbol] , denoted [symbol] , of dimension J₁× . . . ×J_{Q}, the various modes of which correspond to the commands of various effectors (or to the various degrees of freedom of a multi-axis robot).

More specifically, the output tensor provides command data blocks, each block making it possible to generate the command signals relating to the various effectors or degrees of freedom. Thus, it will be understood that the dimension of each data block may depend on the use case envisaged and particularly on the number of degrees of freedom of the effector. Without loss of generality, it will be assumed in the following that the command tensor [symbol] is of order Q=1. In other words, [symbol] [symbol] where M is the number of degrees of freedom of the command (or of the effector).

The predictive model making it possible to change from the observation tensor to the command tensor may be based on a classification and/or a regression. In the case of a classification, the command tensor may indicate for example a movement direction (left, right, front, back), in the case of a regression the command tensor may give the command data of the various effectors.

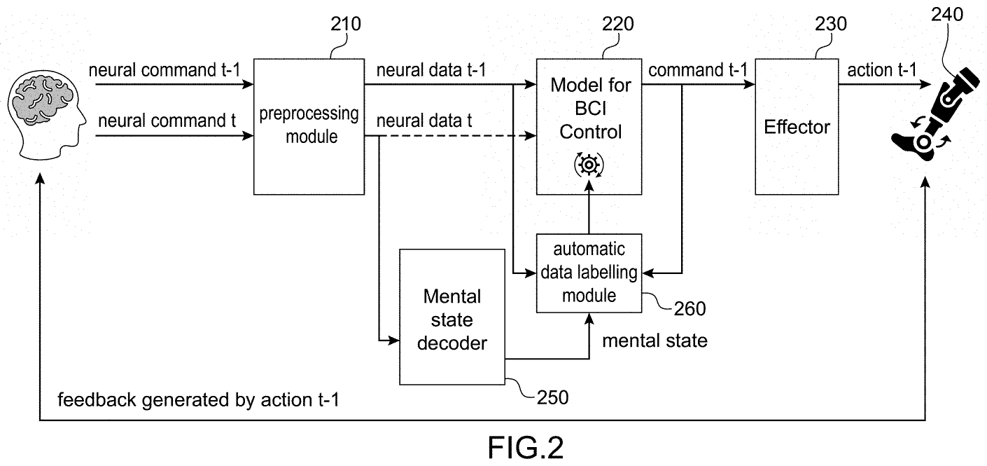

FIG. 2 schematically represents the operation of an adaptive brain computer interface according to one embodiment of the present invention using a first type of architecture.

The elements bearing the references 210 to 240 are identical to the elements 110 to 140 described above. The BCI represented further comprises a mental state decoder 250, trained beforehand, receiving the observation data tensor, [symbol] at the epoch t and estimating from this tensor a state vector representative of the mental state of the subject at this same instant. Mental state at an instant t, means here a satisfaction state or an error state detected from electrophysiological signals (typically ECoG signals) collected by electrodes placed on the motor cortex of the subject. More specifically, this mental state indicates if the subject is satisfied or not of the evolution of the trajectory (of a cursor, of an effector or of a multi-axis robot for example) in other words if the command tensor [symbol] produced by the predictive model and applied to the effector at the instant t−1 is indeed in accordance with the setpoint trajectory desired by the subject.

It is important to clearly make the distinction here between the decoding of an error mental state in the context of the present invention, on the one hand, and the detection of an error potential, on the other hand. An error potential or ErrP (error-related potential) signal is a cerebral signal observed in response to a discrete event, for example an occasional erroneous action. In other words, such an ErrP signal is triggered by an error occurring at a given instant and does not result from a continuous action such as a deviation observed over time in relation to a setpoint trajectory. In practice, the ErrP signals manifest in the form of a negative potential deflection in a fronto-central area of the scalp (appearing approximately 50 to 100 ms after the occurrence of the discrete event), followed by a positive potential deflection in the fronto-parietal area. They may be recorded by simple cutaneous electrodes placed on the scalp whereas the ECoG signals are obtained from electrodes located on the motor cerebral cortex of the subject.

The mental state decoder is trained in a supervised manner during a distinct phase prior to the phase for using the BCI. During this training phase the subject may, for example, be simultaneously presented with a movement setpoint as well as a trajectory. At the same time, the observation data tensors output from the preprocessing module are stored in a mental state training database.

If the trajectory is in accordance with (or tends to move closer to) the setpoint, the observation data tensor is labelled as a satisfaction mental state of the setpoint. Conversely, when the trajectory is not in accordance with (or tends to move away from) the setpoint, the observation data tensor is labelled as an error mental state. In a particularly simple example of embodiment, the setpoint may be a start/stop command. Thus, an avatar may be shown on a screen simultaneously with a symbol indicating the setpoint. If the avatar starts when the setpoint is a stop instruction or if the avatar is immobile when the setpoint is a start instruction, the corresponding observation data tensors are labelled with an error mental state label. On the other hand, if the avatar starts and stops according to the instructions given by the setpoint, the observation data tensors are labelled with a satisfaction mental state label. Of course, other mental state training types may be envisaged by the person skilled in the art according to the nature of the command, without in as much departing from the scope of the present invention. Thus, the setpoint may be a direction instruction (left, right, front, back), or also an instruction indicating the limb to move (left foot, right foot, right hand, left hand). When the movement of the avatar is in accordance with the instruction given by the setpoint, the mental state label associated with the observation data tensors corresponds to a satisfaction mental state. Failing this, when the movement of the avatar differs from the instruction given by the setpoint, the mental label associated with the observation data tensors corresponds to an error mental state. The satisfaction/error mental state at the instant t may be represented by a signed binary value or a Boolean (classifier in 2 classes), denoted y_{D,mental_state}. The training data set of the mental state decoder then consists of the pairs ( [symbol] , \tilde{y}_{D,mental_state}^{t}) in a plurality of observation instants t (the tilde sign indicates the fact that this involves training data).

The error state decoder may be for example implemented by means of an artificial neural network or a SVM classifier, or even an algorithm of the NPLS type.

After its training phase, the mental state decoder 250 may predict the satisfaction/error mental state from an observation data tensor, [symbol] .

According to a first variant, the satisfaction/error mental state predicted at the instant t by the decoder 250 is in the form of a binary value, denoted \tilde{y}_{D,mental_state}^{t}. For example, a satisfaction mental state will be indicated by \tilde{y}_{D,mental_state}^{t}=1 and an error mental state will be indicated by \tilde{y}_{D,mental_state}^{t}=−1.

According to a second variant, the satisfaction/error mental state predicted at the instant t by the decoder 250 is in the form of a real value, denoted \tilde{y}_{mental_state}^{t} indicating the probability that the mental state belongs to one class rather than to another. For example, the real value may be a ratio logarithm of the probability of belonging to one class rather than to another. Thus, a positive value of \tilde{y}_{mental_state}^{t} may translate a satisfaction mental state and a negative value \tilde{y}_{mental_state}^{t} may then translate an error mental state, the degree of certainty of the forecast being given in both cases by |\tilde{y}_{mental_state}^{t}|.

During the use of the BCI, the mental state decoder may provide each epoch t with a prediction of the mental state of the subject from the observation data tensor [symbol] . This mental state prediction is used by an automatic data labelling module 260, to construct on the fly new training data from the pair formed by the observation data tensor and from the command data tensor at the preceding epoch, namely ( [symbol] , [symbol] ).

This creation of training data is not generally systematic at each epoch but may occur during training phases occurring periodically or asynchronously. Without loss of generality, it will be assumed that a training phase of index u starts at the epoch n(u−1)+1 and ends at the epoch nu. The observation data may be represented by the observation tensors [symbol] at the consecutive instants t=n(u−1)+1, . . . nu and therefore by a tensor [symbol] of order P+1=4, [symbol] ∈ [symbol] , such as [symbol] = [symbol] t=n(u=1)+1, . . . nu where [symbol] represents the deployment of [symbol] according to the first mode. Similarly, the command data at these same instants may be represented by a tensor of order Q+1=2, [symbol] ∈ [symbol] such as [symbol] = [symbol] , t=n(u−1)+1, . . . nu where [symbol] represents the deployment of [symbol] according to the first mode. Finally, \tilde{y}_{mental_state}^{u} represents the tensor of order 1, in other words the vector of [symbol] ⁿ the elements of which are \tilde{y}_{mental_state}^{t}, t=n(u−1)+1, . . . nu.

Generally, at each training phase, the automatic labelling module automatically constructs training data defined by the pair ( [symbol] , [symbol] ), such that:

[Math. 1]

[symbol]

=Φ( [symbol] ,\tilde{y}_{mental_state}^{u}) (1-1)

[Math. 2]

[symbol]

= [symbol] (1-2)

where Φ is an application of [symbol] × [symbol] → [symbol] . More specifically, to automatically construct training data, the automatic labelling module uses the observation data tensors of the phase u and associates with them the command data tensors modified by the function Φ when the mental states observed during this phase comprise at least one error mental state. More specifically, the modification of a command tensor at an epoch t_{c} of the phase u may depend on the mental states predicted at instants t_{c}+1, . . . ,nu+1 or even also on states before t_{c}.

Without loss of generality, it will be assumed in the following that a command tensor at an epoch t only depends on the mental state predicted at the following instant t+1. In other words, when the subject receives sensory feedback at the instant t+1 (correction or error of the trajectory) after the command data tensor has been applied, the labelling module modifies (or corrects) the command data tensor relating to the preceding instant, t, which may be expressed by:

[Math. 3]

[symbol]

= [symbol] (2-1)

[Math. 4]

[symbol]

=φ( [symbol] ,(\tilde{y}_{mental_state}^{t+1})t=n(u−1)+1, . . . nu (2-2)

where φ is an application of [symbol] × [symbol] × [symbol] .

The application φ (or plus generally the application Φ) may take various forms depending on the type of prediction made by the predictive model 210. In any case, its object is to update the training data set with the pair ( [symbol] , [symbol] ) thanks to the predicted satisfaction/error mental state, \tilde{y}_{mental_state}^{t+1} at at least the following instant of the training phase. If the mental state predicted for at least this following instant corresponds to an error mental state, the command data tensor [symbol] is corrected by the application φ to generate the new training data ( [symbol] , [symbol] ). On the other hand, if all the earlier mental states \tilde{y}_{mental_state}^{t=1}, . . . , \tilde{y}_{mental_state}^{nu+1} are satisfaction states, the pair may be ( [symbol] , [symbol] ) may be incorporated as is in the training data set.

According to a first embodiment, the prediction made by the predictive model is based on a classification operation providing a vector y_{class}=(y₁^{t}, y₂^{t}, . . . y_{M}^{t})^{T} of probabilities of belonging to M possible classes, and the command vector ( [symbol] ) provided by the predictive model is given by:

$$\begin{matrix}[ Math . 5 ] & \\ {y}_{control}^{t} = {e}_{{m}_{1}^{t}} & ( 3 )\end{matrix}$$

where

$${m}_{1}^{t} = {arg max}_{m = 1 , . . . , M} ( {y}_{m}^{t + 1} )$$

and (eₘ)_{m-1, . . . ,M} is the canonical basis of [symbol] . In other words the command vector corresponds to the class of highest probability.

The automatic labelling module updates the training data set by incorporating the pair ( [symbol] , [symbol] )=( [symbol] ,\tilde{y}^{t}) defined by:

[Math. 6]

[symbol]

= [symbol] (4-1)

[Math. 7]

\tilde{t}¹=φ(y_{control}^{t},\tilde{y}_{mental_state}^{t+1}) (4-2)

where:

φ(y_{control}^{t},\tilde{y}_{mental_state}^{t+1})=y_{control}^{t} (5-1)

if the mental state is a satisfaction mental state (\tilde{y}_{D,mental_state}^{t+1}=+1 or \tilde{y}_{mental_state}^{t+1}<0); and

$$\begin{matrix}[ Math . 8 ] & \\ φ ( {y}_{control}^{t} , {\hat{y}}_{mental\_state}^{t + 1} ) = {T}_{{m}_{1}^{t} {m}_{2}^{t}} {y}_{control}^{t} = {e}_{{m}_{2}^{t}} & ( 5 \text{-} 2 )\end{matrix}$$

if the mental state is an error mental state

$$\begin{matrix}[ Math . 8 ] & \\ φ ( {y}_{control}^{t} , {\hat{y}}_{mental \_ state}^{t + 1} ) = {T}_{{m}_{1}^{t} {m}_{2}^{t}} {y}_{control}^{t} = {e}_{{m}_{2}^{t}} & ( \text{5-2)}\end{matrix}$$

Tₘₜₘ_{₂}ₜ being the size array M×M permuting the lines m₁^{t} and m₂^{t} with

$${m}_{2}^{t} = {arg max}_{{m = 1 , . . . , M}_{m \neq {m}_{1}^{t}}} ( {y}_{m}^{t + 1} ) .$$

In other terms, if the mental state at the following instant is an error state, the command vector is given by the second most probable class.
According to one variant, the incorporation of new training data is selective. More specifically, in this case, a pair ( [symbol] , [symbol] ) will only be incorporated into Ω^{u} insofar as the degree of certainty of the satisfaction mental state in (5-1) exceeds a predetermined threshold value, that is to say if \tilde{y}_{mental_state}^{t+1}<Th_{mental_state}¹<0. Similarly, the correction made in (5-2) may also be selective and only be performed insofar as \tilde{y}_{mental_state}^{t+1}<Th_{mental_state}²<0 where Th_{mental_state}² is a second predetermined threshold value.
The predictive model 220 is updated by means of new training data provided by the automatic labelling module. This update does not necessarily occur the moment that these new training data are available. Indeed, the latter may be stored locally for a later update, performed periodically or as soon as the number of new training data reaches a predetermined threshold.
The update of the parameters of the predictive model is performed by minimising a cost function giving the square deviation between the predictions of the model and the labels for the data of the training set, i.e.:

$$\begin{matrix}[ Math . 9 ] & \\ {Θ}^{u} = {arg min}_{Θ} {\sum }_{( {\tilde{\underline{X}}}^{t} , {\tilde{\underline{Y}}}^{t} ) ∈ {Ω}^{u}} {‖ F ( \tilde{{\underline{X}}^{t}} ; Θ ) - \tilde{{Y}^{t}} ‖}^{2} & ( 6 )\end{matrix}$$

where Θ designates the set of parameters of the predictive model, Θ^{u} designates the set of parameters minimising the cost function during the update u, F(·;Θ) is the prediction function of the model depending on the set of parameters Θ, Ω^{u}=Ω^{u-1}∪{( [symbol] , [symbol] ); t=(u−1)n+1, . . . , un } is the training data set during the update u.

The cost function may involve a weight depending on the degree of certainty of the mental state prediction to weight the prediction square deviation of the command tensor, i.e.:

$$\begin{matrix}[ Math . 10 ] & \\ {Θ}^{u} = {arg min}_{Θ} {\sum }_{( {\tilde{\underline{X}}}^{t} , {\tilde{\underline{Y}}}^{t} ) ∈ {Ω}^{u}} w ( {\hat{y}}_{mental \_ state}^{t + 1} ) {‖ F ( \tilde{{\underline{X}}^{t}} ; Θ ) - \tilde{{Y}^{t}} ‖}^{2} & ( 7 )\end{matrix}$$

where w(\tilde{y}_{mental_state}^{t+1}) is an increasing function of the degree of certainty for \tilde{y}_{mental_state}^{t+1}>0, in other words when the mental state is a satisfaction mental state. In other terms, the cost function will give more weight to training data having a higher probability of corresponding with a satisfaction mental state.

The following may be taken for example:

[Math. 11]

w(\tilde{y}_{mental_state}^{t+1})=max(\tilde{y}_{mental_state}^{t+1}−−Th_{mental_state}¹,0) (8-1)

or

[Math. 12]

w(\tilde{y}_{mental_state}^{t+1})=h(\tilde{y}_{mental_state}^{t+1}−−Th_{mental_state}¹) (8-2)

where h(·) is the Heaviside step. It will be noted that the choice of a weighting function according to (8-2) is equivalent to the selective incorporation into the training set according to the aforementioned variant.

According to a second embodiment, the prediction made by the predictive model is based on a regression operation, for example a linear or multilinear regression:

[Math 13]

[symbol]

= [symbol] + [symbol] (9)

where [symbol] is a prediction coefficient tensor [symbol] ∈ [symbol] and [symbol] is a skew tensor [symbol] ∈ [symbol] . The set of parameters of the predictive model here consists of the coefficients of the tensors [symbol] and [symbol] , Θ={ [symbol] , [symbol] }. The tensor [symbol] provided by the predictive model is used directly for the command.

Alternatively, the prediction may be performed by means of a non-linear regression for example by means of an artificial neural network.

Regardless of the regression type, according to a first variant, the automatic labelling module updates the training data set Ω^{u} by incorporating thereto the pair ( [symbol] , [symbol] )=( [symbol] , [symbol] ) if the degree of certainty of the satisfaction mental state exceeds a predetermined threshold value (\tilde{y}_{mental_state}^{t}>Th_{mental_state}¹). Failing this, the labelling is not carried out and the pair ( [symbol] , [symbol] ) is not incorporated into Ω^{u}.

As in the first embodiment, the update of the parameters of the predictive model may be performed by minimising a cost function giving the square deviation between the predictions of the model and the labels for the data of the training set:

$$\begin{matrix}[ Math . 14 ] & \\ {Θ}^{u} = {arg min}_{Θ} {\sum }_{( {\tilde{\underline{X}}}^{t} , {\tilde{\underline{Y}}}^{t} ) ∈ {Ω}^{u}} {‖ F ( \tilde{{\underline{X}}^{t}} ; Θ ) - \tilde{{Y}^{t}} ‖}^{2} & ( 10 )\end{matrix}$$

where) F(·;Θ) is the regression function.

According to a second alternative embodiment, the automatic labelling module updates the training data set Ω^{u} by incorporating thereto the pair ( [symbol] , [symbol] ) and ( [symbol] , [symbol] ) regardless of whether the predicted mental state is a satisfaction mental state or an error state. In this case, the update of the parameters on the training set is done by minimising a cost function giving more weight to the training data that correspond to a mental state having a higher degree of certainty (regardless of whether this mental state is a satisfaction or error mental state) than to the training data for which the predicted mental state is uncertain, namely:

$$\begin{matrix} [ Math . 15 ] & \\ {Θ}^{u} = {arg min}_{Θ} {\sum }_{( {\tilde{\underline{X}}}^{t} , {\tilde{\underline{Y}}}^{t} ) ∈ {Ω}^{u}} exp ( {\hat{y}}_{D , mental \_ state}^{t + 1} \cdot w ( {\hat{y}}_{mental \_ state}^{t + 1} ) \cdot {‖ F ( \tilde{{\underline{X}}^{t}} ; Θ ) - \tilde{{Y}^{t}} ‖}^{2} ) & ( 11 )\end{matrix}$$

with:

[Math. 16]

w(\tilde{y}_{mental_state}^{l+1})=|\tilde{y}_{mental_state}^{l+1}| if \tilde{y}_{menatl_state}^{t+1}≤Th₋ or \tilde{y}_{mental_state}^{t+1}≥Th₊ (12-1)

and

[Math. 17]

w(\tilde{y}_{mental_state}^{l+1})=0 if Th₋<\tilde{y}_{mentaal_state}^{t-1}<Th₊ (₁₂-₂)

where Th₋ and Th₊ are respectively a negative threshold value and a positive threshold value.

Due to the presence of the signed binary value, \tilde{y}_{D,mental_state}^{t+1}, in the expression (11), the minimisation of the cost function tends to reduce the square deviation of the prediction on the training data corresponding to a satisfaction mental state and to increase this deviation on the training data corresponding to an error mental state. The contribution to the reduction or to the increase of the square deviation depends on the degree of certainty of the prediction of the mental state, |\tilde{y}_{mental_state}^{t+1}|.

Equivalently, the consideration of a zero weight in the expression (12-2) may be implemented by only incorporating into the training data set the pairs of tensors ( [symbol] , [symbol] ) for which the degree of certainty of the predicted mental state, |\tilde{y}_{mental_state}^{t+1}|, is sufficiently high.

The update of the parameters of the model depends on the type of predictive model. For example, if the predictive model is produced by means of a neural network the update of the parameters may be conventionally obtained by back propagation of the gradient. When the predictive model is based on a linear or multilinear regression, the update of the parameters may be performed according to the REW-PLS (Recursive Exponentially Weighted Partial Least Squares) or REW-NPLS (Recursive Exponentially Weighted N-way Partial Least Squares) algorithm, the cost function minimisation then being applied at each step of the Alternate Least Squares (ALS) method of the PARAFAC breakdown.

A description of the REW-PLS and REW-NPLS algorithms may be found in the article by A. Eliseyev et al. entitled “Recursive exponentially weighted N-way Partial Least Squares regression with recursive validation of hyper-parameters in Brain-Computer Interface applications” published in Scientific Reports, vol. 7, no. 1, p. 16281, November 2017 as well as in the patent application FR-A-3 061 318. These algorithms are advantageous insofar as they do not need to store the history of the training data but only those that have been labelled since the last update.

In the embodiment of FIG. 2, the module 220 implementing the predictive model, that is to say computing the command tensor from the observation tensor, is also responsible for updating the parameters of the model. For this purpose, it locally stores the training data provided by the automatic labelling module 260. The update of the parameters may be performed at the same time with the computation of the command tensor, by multithreading in a central processing unit (CPU).

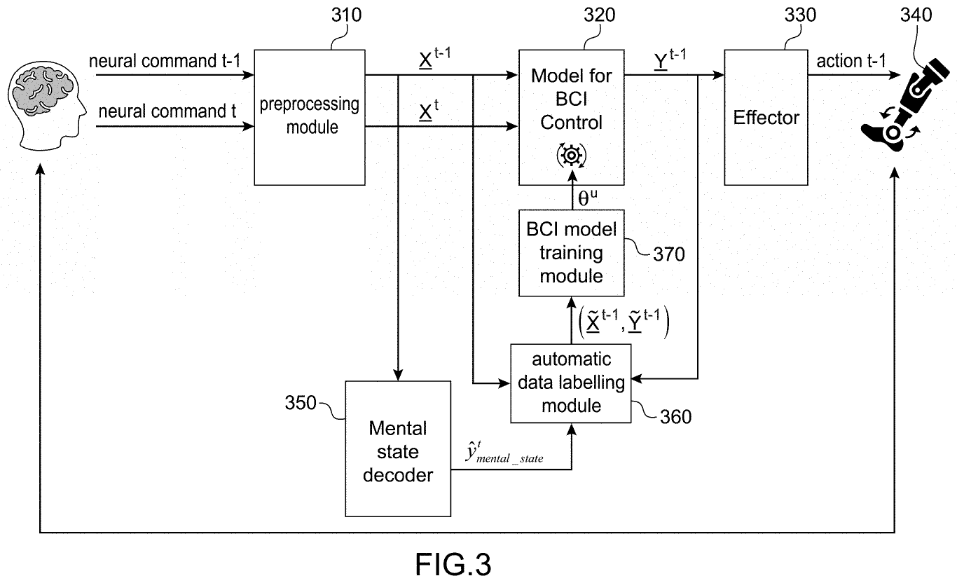

FIG. 3 schematically represents the operation of an adaptive brain computer interface according to one embodiment of the present invention using a second type of architecture.

The elements 310, 330, 340, 350 and 360 are respectively identical to the elements 210, 230, 240, 250 and 260 of FIG. 2 and their description will therefore not be repeated here.

The adaptive brain computer interface of FIG. 3 differs from that of FIG. 2 in that it comprises a module for training the predictive model, 370, distinct from the module implementing the predictive model itself. In other words, the module 320 makes a prediction (classification or regression) of the command data tensor [symbol] from the observation data tensor [symbol] by means of the prediction function F(·;Θ) but itself does not carry out the update of the parameters Θ. This is delegated to the training module 370 that receives the training data from the automatic labelling module 360. For example, when a new set of parameters Θ^{u} is available at the end of a new training phase u, the training module notifies this to the prediction module by means of an interruption to its CPU. The prediction module may then download the new set of parameters without disturbing any parameters of the command.

The person skilled in the art will understand that the brain computer interface described above is adaptive insofar as it adapts to the non-stationary condition of neural signals. It does not require a dedicated training phase, the training being able to be performed on training data obtained by an automatic labelling process using a prediction of the satisfaction/error mental state of the user. Furthermore, the labelled data correspond to tasks that the user actually carries out and not to tasks that are imposed on them during supervised training. Finally, it should be noted that the automatic labelling of observation data makes it possible to generate significant amounts of labelled data that may be used in an off-line training method. Thus, training databases can be obtained by crowd-sourcing without needing long expensive and demanding training sessions for the user.

1. A method for training a brain computer interface configured to receive a plurality of electrophysiological signals expressing a neural command of a subject, during a plurality of observation windows associated with observation instants, he electrophysiological signals being preprocessed in a preprocessing module to form at each observation instant an observation data tensor, the brain computer interface using a predictive model to deduce at each observation instant a command data tensor from the observation data tensor, the command data being configured to control at least one effector to perform a trajectory, the training method comprising:
at each observation instant, decoding a satisfaction/error mental state of the subject from the observation data tensor using a mental state decoder trained beforehand, the mental state being representative of a conformity of the trajectory with the neural command;
generating training data from the satisfaction/error decoded at a given observation instant, and from a pair formed by the observation data tensor and the command data tensor at a preceding observation instant; and
updating parameters of the predictive model by minimising a cost function on the generated training data.

2. The method for training a brain computer interface according to claim 1, comprising training the mental state decoder in a previous phase by presenting simultaneously to the subject a movement setpoint and a trajectory, the observation data tensor being labelled with a satisfaction mental state when the trajectory is in accordance with the setpoint and with an error mental state when it deviates therefrom.

3. The method for training a brain computer interface according to claim 2, wherein the mental state decoder provides at each observation instant a prediction of the mental state in a form of a binary value (\tilde{y}_{D,mental_state}^{t}) as well as an estimation of a degree of certainty of the prediction (|\tilde{y}_{mental_state}^{t}|).

4. The method for training a brain computer interface according to claim 3, wherein the prediction made by the predictive model is based on a classification, the command data tensor being obtained from a most probable class predicted by the predictive model.

5. The method for training a brain computer interface according to claim 4, comprising, if the mental state predicted at an observation instant is a satisfaction state, generating the training data only from the observation data tensor and from the command data tensor at the preceding observation instant, if the degree of certainty of the predicted mental state is greater than a first predetermined threshold value.

6. The method for training a brain computer interface according to claim 4, comprising, if the mental state predicted at an observation instant is an error state, generating the training data only from the observation data tensor and from the command data tensor at the preceding observation instant, if the degree of certainty of the predicted mental state is greater than a second predetermined threshold value.

7. The method for training a brain computer interface according to claim 4, wherein if the mental state predicted at an observation instant is an error state, the training data generated comprise the observation data tensor at the preceding observation instant as well as a command data tensor obtained from a second most probable class predicted by the predictive model at the preceding observation instant.

8. The method for training a brain computer interface according to claim 4, wherein the cost function used for updating the parameters of the predictive model expresses a square deviation between the command data tensor predicted by the model and that provided by the training data, the square deviation being weighted by a degree of certainty predicted by the mental state decoder during the generation of the training data, the square deviation thus weighted being added to the training data set.

9. The method for training a brain computer interface according to claim 3, wherein the prediction made by the predictive model is based on a linear or multilinear regression.

10. The method for training a brain computer interface according to claim 9, wherein if the mental state predicted at an observation instant is an error state, the training data are not generated and that if the predicted mental state is a satisfaction state, the training data are only generated from the observation data tensor and from the command data tensor at the preceding observation instant, if the degree of certainty of the predicted mental state is greater than a first predetermined threshold value.

11. The method for training a brain computer interface according to claim 9, wherein regardless of the state predicted at an observation instant, the training data are generated from the observation data tensor and from the command data tensor at the preceding observation instant, the training data then being associated with the degree of certainty of the prediction of the predicted mental state (|\tilde{y}_{mental_state}^{t}|).

12. The method for training a brain computer interface according to claim 9, wherein the cost function used for updating the parameters of the predictive model depends on a square deviation between the command data tensor predicted by the predictive model and that provided by the training data, the dependency with the square deviation being increasing when the mental state predicted during the generation of the training data was a satisfaction state and decreasing when the mental state is an error signal, the square deviation being weighted by a factor depending increasingly on the degree of certainty of the predicted mental state, associated with the training data.
